(19) United States
(12) Patent Application Publication
(10) Pub. No.: US 2024/0060901 A1
(43) Pub. Date: Feb. 22, 2024

(54) SIGNAL ENHANCEMENT STRUCTURE AND MEASURING METHOD WITH SIGNAL ENHANCEMENT

(71) Applicants: National Chung Hsing University, Taichung City (TW); PROTRUSTECH CO., LTD, Tainan City (TW)

(72) Inventors: Chien-Chung Chang, Taichung City (TW); Chun-Ta Huang, Tainan City (TW)

(73) Assignees: National Chung Hsing University, Taichung City (TW); PROTRUSTECH CO., LTD, Tainan City (TW)

(21) Appl. No.: 18/496,942

(22) Filed: Oct. 30, 2023

Related U.S. Application Data
(63) Continuation-in-part of application No. 17/366,029, filed on Jul. 2, 2021.

(30) Foreign Application Priority Data
Jul. 3, 2020 (TW) 109122573

(51) Int. Cl. G01N 21/65; G01N 21/64; B22F 1/054

(52) U.S. Cl. CPC G01N 21/658; G01N 21/648; B22F 1/0547

(57) ABSTRACT

A signal enhancement structure configured to enhance a signal of a specimen is provided. The signal enhancement structure includes a plurality of nanowires stacked in a first direction, a second direction, and a third direction. The nanowires are extended along at least two directions. A particle of the specimen is on the nanowires or in a gap among the nanowires. A manufacturing method of a signal enhancement structure and a measuring method with signal enhancement are also provided.

### CROSS-REFERENCE TO RELATED APPLICATION

This application is a continuation-in-part application of and claims the priority benefit of U.S. application Ser. No. 17/366,029, filed on Jul. 2, 2021, which claims the priority benefit of Taiwan application serial no. 109122573, filed on Jul. 3, 2020. The entirety of each of the above-mentioned patent applications is hereby incorporated by reference herein and made a part of this specification.

### BACKGROUND OF THE INVENTION

### Field of the Invention

The invention relates to a signal enhancement structure, a manufacturing method thereof, and a measuring method with signal enhancement.

### Description of Related Art

Raman spectroscopy is a type of vibrational spectroscopy. The principle thereof is to use a laser light source with a fixed wavelength to excite a sample. When the excitation light interacts with a sample particle, if the photon exchanges energy after colliding with the particle, then the photon transfers a portion of the energy to the sample particle or obtains a portion of the energy from the sample particle, thereby changing the frequency of the light. This change is called the Raman shift.

The measurement of Raman spectroscopy has the advantages that the sample may be detected to obtain results in real time without pretreatment and without damage. In addition, Raman spectroscopy may be analyzed by microscopy, and the resolution thereof may reach the sub-micron level, making the analysis more accurate. In addition, Raman spectroscopy may also have the advantages of high selectivity, high sensitivity, and high mobility. Raman spectroscopy may be used for food testing, biomedical testing, environmental testing, and drug testing, etc. Moreover, photoluminescence spectroscopy, especially fluorescence spectroscopy, and may also be used for various detections without sample pretreatment and without sample damage.

Sometimes the signal of Raman spectroscopy or photoluminescence spectroscopy is not strong enough, and it is more difficult to produce good detection results.

### SUMMARY OF THE INVENTION

The invention provides a signal enhancement structure for detecting objects of different particle sizes.

The invention provides a manufacturing method of a signal enhancement structure for detecting objects of different particle sizes.

An embodiment of the invention provides a signal enhancement structure configured to enhance a signal of a specimen. The signal enhancement structure includes a plurality of nanowires stacked in a first direction, a second direction, and a third direction. The nanowires are extended along at least two directions. A particle of the specimen is on the nanowires or in a gap among the nanowires.

An embodiment of the invention provides a manufacturing method of a signal enhancement structure, including the following steps. A plurality of nanowires dispersed in a solvent are sprayed on a surface to form a first nanowire layer; and after the solvent in the first nanowire layer is volatilized, the plurality of nanowires dispersed in the solvent are sprayed on the first nanowire layer again to form a second nanowire layer.

An embodiment of the invention provides a signal enhancement structure configured to enhance a signal of a specimen. The signal enhancement structure includes a plurality of nanowires stacked in a first direction, a second direction, and a third direction. The nanowires are extended along at least two directions. An included angle of the nanowires is varied in planes perpendicular to the first direction, the second direction, and the third direction, and a particle of the specimen is on the nanowires or in a gap among the nanowires or the nanowires are on the specimen. The nanowires are stacked in the third direction to form a film layer. The third direction is a thickness direction of the film layer. The first direction and the second direction are both perpendicular to the third direction, and a thickness of the film layer in the third direction ranges from 350 nanometers to 550 nanometers.

An embodiment of the invention provides a measuring method with signal enhancement including: dropping or spraying a plurality of nanowires dispersed in a solvent on a surface of a specimen to form a film layer on the specimen, wherein the film layer has a thickness ranging from 350 nanometers to 550 nanometers; and measuring at least one of a Raman spectrum and a photoluminescence spectrum of the specimen, a signal of which is enhanced by the nanowires.

An embodiment of the invention provides a measuring method with signal enhancement including: mixing a specimen with a plurality of nanowires dispersed in a solvent; dropping or spraying the specimen with the nanowires dispersed in the solvent onto a substrate to form a film layer on the substrate, wherein the film layer has a thickness ranging from 350 nanometers to 550 nanometers; and measuring at least one of a Raman spectrum and a photoluminescence spectrum of the specimen, a signal of which is enhanced by the nanowires.

An embodiment of the invention provides a measuring method with signal enhancement including: dropping or spraying a plurality of nanowires dispersed in a solvent on a substrate to form a film layer on the substrate, wherein the film layer has a thickness ranging from 350 nanometers to 550 nanometers; dropping or spraying a specimen onto the film layer; and measuring at least one of a Raman spectrum and a photoluminescence spectrum of the specimen, a signal of which is enhanced by the nanowires.

In the signal enhancement structure of an embodiment and the manufacturing method thereof according to the invention, since the nanowires are stacked in the first direction, the second direction, and the third direction, the particle of the specimen may have different distances from different nanowires, such that the signal of the particle of the specimen may be enhanced. Therefore, the signal enhancement structure of an embodiment of the invention is suitable for specimens of different sizes. In the signal enhancement structure and the measuring method with signal enhancement according to the embodiment of the invention, since the thickness of the film layer ranges from 350 nanometers to 550 nanometers, the signal enhancement is more significant.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a diagram of a signal enhancement structure of the invention for detecting coronavirus.

FIG. 2A is a three-dimensional diagram of the signal enhancement structure of an embodiment of the invention.

FIG. 2B is a top view of the signal enhancement structure of FIG. 2A.

FIG. 2C is a three-dimensional diagram of the signal enhancement structure of another embodiment of the invention.

FIG. 3 is a three-dimensional diagram illustrating surface plasmon resonance generated by the signal enhancement structure of FIG. 2A.

FIG. 4 is a top view of the signal enhancement structure of another embodiment of the invention.

FIG. 5 is a three-dimensional diagram of the signal enhancement structure of another embodiment of the invention.

FIG. 6 is a three-dimensional diagram of the signal enhancement structure of yet another embodiment of the invention.

FIG. 7 is a three-dimensional diagram of the signal enhancement structure of another embodiment of the invention.

FIG. 8 is a cross-sectional view for explaining the manufacturing method of a signal enhancement structure of an embodiment of the invention.

FIG. 9 is a diagram of the optical path architecture of a spectrum measurement system of an embodiment of the invention.

FIG. 10A and FIG. 10B respectively show the theoretical electric field values and experimental SERS results of multilayer nanostructured silver nanowires.

FIG. 11 is a schematic structural view showing a step of a method with signal enhancement according to an embodiment of the invention.

FIG. 12 is a schematic structural view showing a step of a method with signal enhancement according to another embodiment of the invention.

FIG. 13A and FIG. 13B are schematic structural views showing two steps of a method with signal enhancement according to another embodiment of the invention.

### DESCRIPTION OF THE EMBODIMENTS

The invention provides a signal enhancement structure and method that may be used to enhance both “surface enhanced Raman scattering” (SERS) and “metal enhanced fluorescence” (MEF) to compensate for insufficiencies in the traditional use of Raman signal or fluorescent signal for detection. SERS is a technique that enhances the sensitivity of Raman scattering by adsorbing particles or plasmas on the surface of nano-grade rough metal, so that the intensity of Raman signals may be increased by several orders of magnitude. However, factors such as different metal materials, the shape and size of surface particles, and the absorption capacity and distance of the probe affect the effect of SERS.

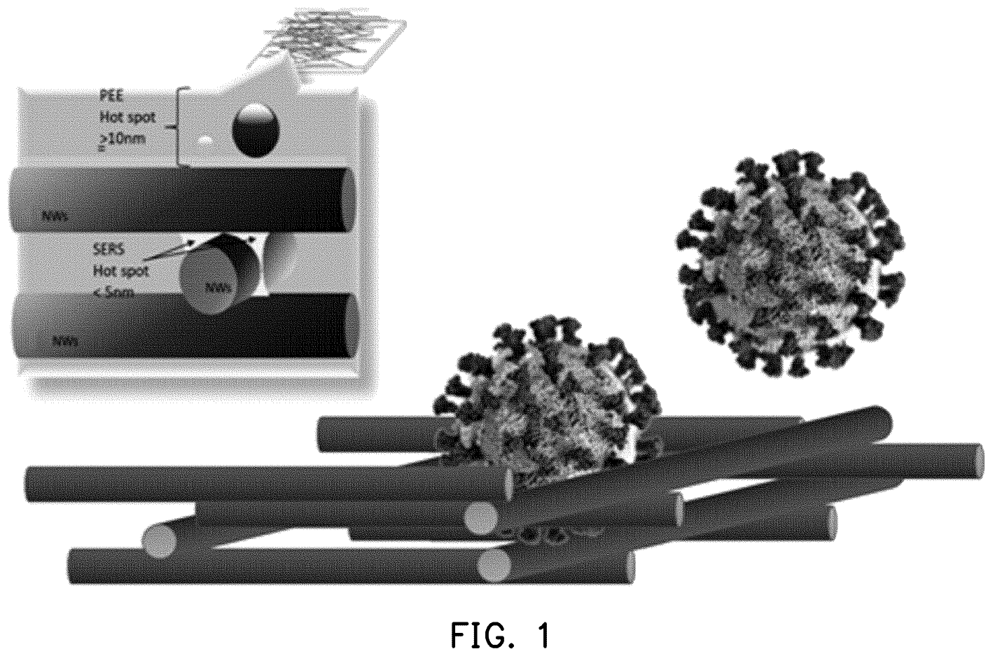

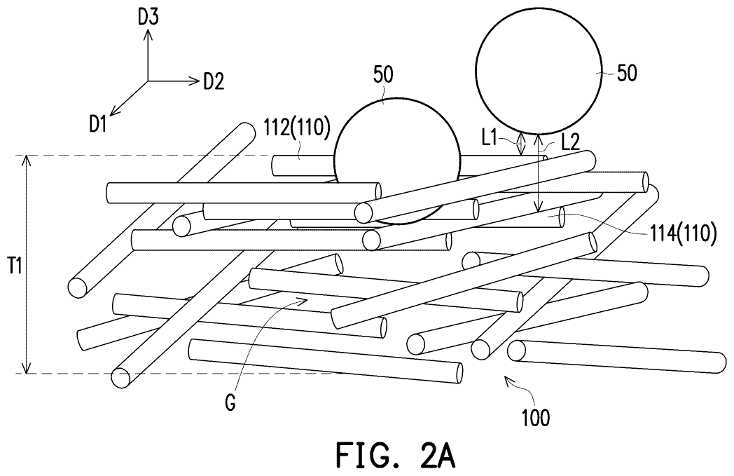

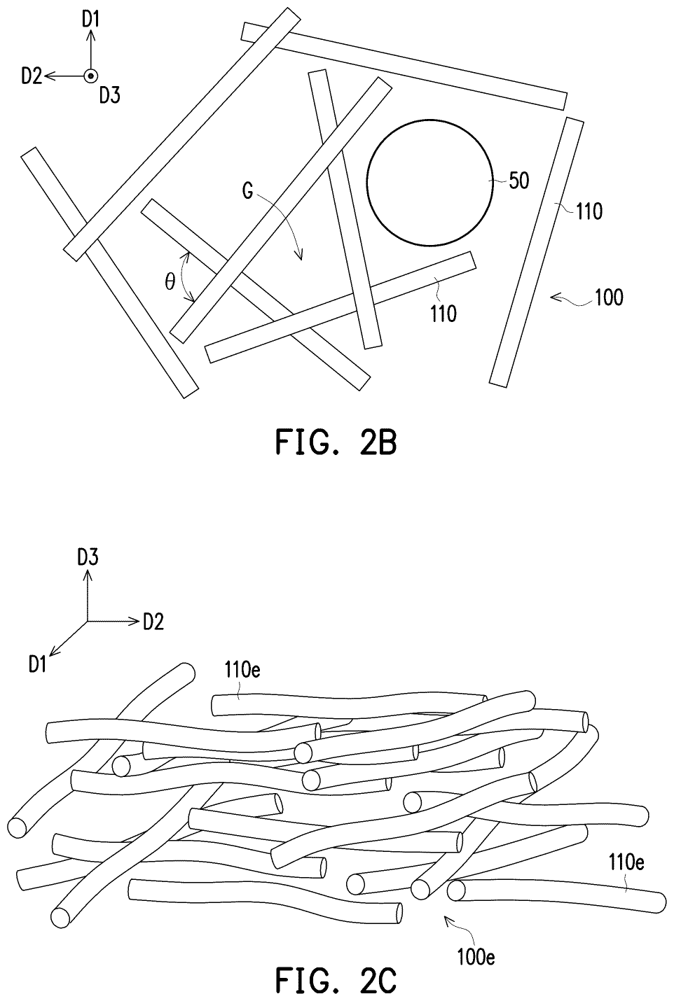

MEF mainly occurs when the spacing between a fluorescent substance and a metal reaches a certain distance (for example, 5 nm to 90 nm). The fluorescent substance is affected by the local electric field of metal nanoparticles, and the excited electrons of the fluorescent substance are affected by the enhanced electromagnetic field enhancement effect, so that more electrons jump to the excited state, thus enhancing the amount of light emitted. The fluorescence enhancement effect is related to the material, shape, and distance of the metal nanoparticles, and the main mechanism thereof is related to the local electric field enhancement near the fluorescent particles of the metal surface. The interaction of the frequency of incident light with the oscillation frequency of metal surface plasma (induced by incident light or fluorescent light emission) causes “localized surface plasmon resonance” (LSPR), and this metal surface plasmon resonance is an important factor that determines the optical properties of metal nanoparticles. FIG. 1 is a diagram of a signal enhancement structure of the invention for detecting coronavirus. The invention mainly uses a stacked nanostructure to enhance the signal of a detection spectrum. For example, the effect of surface enhanced Raman scattering (SERS) or localized surface plasmon resonance (LSPR) may be enhanced, and even the SERS and LSPR signals may be simultaneously or synchronously amplified, thereby increasing the accuracy and application level of detection. Therefore, the structure of the invention may achieve the possibility of detecting a single virus. FIG. 2A is a three-dimensional diagram of a signal enhancement structure of an embodiment of the invention, and FIG. 2B is a top view of the signal enhancement structure of FIG. 2A. Referring to FIG. 2A and FIG. 2B, a signal enhancement structure 100 of the present embodiment is configured to enhance the signal of a specimen, such as a Raman signal or a photoluminescence signal. The signal enhancement structure 100 includes a plurality of nanowires 110 stacked in a first direction D1, a second direction D2, and a third direction D3, wherein the nanowires 110 are extended in at least two directions. Particles 50 of the specimen fall on the nanowires 110 or in a gap G among the nanowires 110. In an embodiment, the particles 50 are, for example, molecules (for example, the outer diameter falls within the range of 1 nanometer to 5 nanometers), nanoparticles (for example, the outer diameter falls within the range of 50 nanometers to 100 nanometers), viruses (for example, the outer diameter is about 120 nanometers), bacteria (for example, the outer diameter is about 500 nanometers to 1000 nanometers), cells (for example, the outer diameter falls within the range of 10000 nanometers to 2000 nanometers), or any combination of the different particles. In the present embodiment, for the nanowires 110 stacked in different directions, the included angles of any two nanowires 110 may be different and varied in the planes perpendicular to the first direction D1, the second direction D2, and the third direction D3. Take FIG. 2B, the view perpendicular to the third direction D3, as an example, the included angles θ of two nanowires 110 are different. For example, some of the angles may be greater than 90 degrees, some may be less than 90 degrees, and some may be equal to 90 degrees. In addition, in the present embodiment, the material of the nanowires includes gold, silver, platinum, other precious metals, or a combination thereof.

In the present embodiment, the nanowires 110 are stacked into a film layer, the third direction D3 is the thickness direction of the film layer, the first direction D1 and the second direction D2 are both perpendicular to the third direction D3, and the particles 50 of the specimen are at different distances from different nanowires 110 in the third direction D3. For example, in FIG. 2A, in the third direction D3, a distance L1 and a distance L2 from the particles 50 of the specimen respectively to the nanowires 112 and to the nanowires 114 are different. In the present embodiment, the nanowires 110 have a straight shape. However, in another embodiment, as shown in FIG. 2C, nanowires 110e of a signal enhancement structure 100e may also have a curved shape. Alternatively, in other embodiments, the nanowires may also be a combination of a curved shape and a straight shape (for example, a mixture of the nanowires 110 of FIG. 2A and the nanowires 110e of FIG. 2C). In the present embodiment, the nanowires 110 are irregularly distributed. When the particles 50 are nanoparticles or viruses, the width ratio of the largest gap (that is, the largest of the gaps G) and the smallest gap (that is, the smallest of the gaps G) among the nanowires 110 falls within the range of 50 to 2000.

For a mechanism of metal enhanced fluorescence, fluorescence enhancement effect is better when the particles of the specimen are kept at a proper distance to the nanostructure, and is worse if the distance is too close or too far. In the signal enhancement structure 100 of the present embodiment, the nanowires 110 are stacked in the first direction D1, the second direction D2, and the third direction D3, that is, the nanowires 110 form a three-dimensional stacked structure.

Therefore, the particles 50 of the specimen may have different distances from different nanowires 110. Therefore, a proper distance is readily kept between the particles 50 and a certain nanowire 110 in the vicinity, so that the signal of the particles 50 of the specimen (such as a fluorescent signal) may be well enhanced. Therefore, the signal enhancement structure 100 of the present embodiment is suitable for the particles 50 of the specimen of various different sizes. In addition, the numerical range of the ratio of the largest gap to the smallest gap is also favorable for the nanowires 110 to carry the particles 50 of the specimen of various different sizes. Therefore, the signal enhancement structure 100 of the present embodiment is suitable for measuring the particles 50 of the specimen of various different sizes. In addition, the measurement of the present embodiment avoids the prior method of binding antibody and antigen to grab the particles of the specimen, thus reducing detection errors effectively. Furthermore, with the signal enhancement structure 100 of the present embodiment, the types of specimen are not limited, so as to detect non-biological molecules (such as pesticides, drugs, etc.), organisms, or organisms thereof (such as bacteria, viruses, etc.), and any specimen generating a Raman signal or a photoluminescence signal.

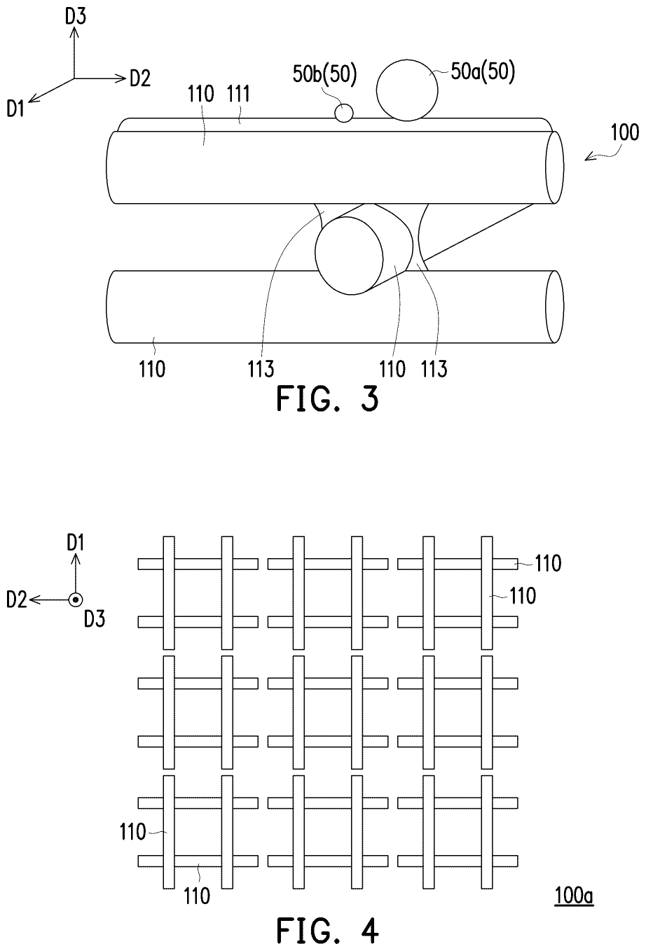

FIG. 3 is a three-dimensional diagram illustrating surface plasmon resonance (SPR) generated by the signal enhancement structure of FIG. 2A. Please refer to FIG. 2A and FIG. 3, when the particles 50 (whether they are larger particles 50a or smaller particles 50b) of the specimen falls on a surface plasma region 111 of the nanowires 110, SERS may be achieved via SPR. In addition, for metal enhanced fluorescence, when the distance between the particles 50 of the specimen and the nanowires 110 is slightly larger than the thickness of the surface plasma region 111 (for example, when the particles 50 in FIG. 3 are on the surface plasma region 111 and a proper distance is kept from the surface plasma region 111), a good metal enhanced fluorescent effect may be achieved. In addition, the nanowires 110 and a region 113 near the intersection of the nanowires 110 (i.e., a SERS hot spot, that is, a plasma distribution region) may achieve good Raman signal enhancement effect via SPR. In other words, the signal enhancement structure 100 of the present embodiment may simultaneously enhance the Raman signal and the photoluminescence signal. In addition, as shown in FIG. 3, the signal enhancement structure 100 of the present embodiment may achieve plasma distribution in the third direction D3 (thickness direction). Therefore, the effect of signal enhancement may be achieved for the particles 50 of the specimen of various different sizes.

Referring to FIG. 2A again, in this embodiment, the nanowires 110 are stacked in the third direction D3 to form a film layer (i.e. the signal enhancement structure 100), and a thickness T1 of the film layer in the third direction D3 ranges from 350 nanometers (nm) to 550 nanometers (nm). When the thickness T1 is within the range of 350 nm to 550 nm, the aforementioned signal enhancement is more significant, and the experimental data are provided hereinafter.

FIG. 4 is a top view of the signal enhancement structure of another embodiment of the invention. Please refer to FIG. 4, a signal enhancement structure 100a of the present embodiment is similar to the signal enhancement structure 100 of FIG. 2B, and the difference between the two is that the nanowires 110 of the signal enhancement structure 100a are regularly distributed, such as arranged in various geometric shapes, and the invention is not limited thereto.

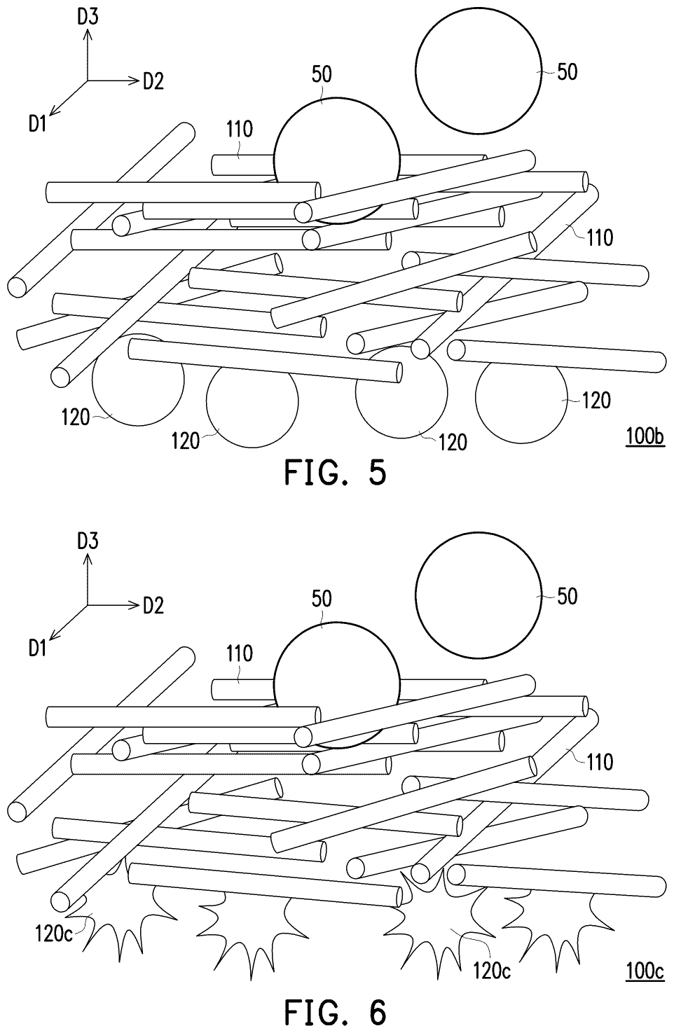

FIG. 5 is a three-dimensional diagram of the signal enhancement structure of another embodiment of the invention. Referring to FIG. 5, a signal enhancement structure 100b of the present embodiment is similar to the signal enhancement structure 100 of FIG. 2A, and the differences between the two are as follows. The signal enhancement structure 100b of the present embodiment further includes a plurality of nanoparticles 120, and the nanowires 110 are substantially stacked on the nanoparticles 120. The material of the nanoparticles 120 is, for example, gold, silver, platinum, other precious metals, or a combination thereof.

FIG. 6 is a three-dimensional diagram of the signal enhancement structure of yet another embodiment of the invention. Referring to FIG. 6, a signal enhancement structure 100c of the present embodiment is similar to the signal enhancement structure 100 of FIG. 2A, and the differences between the two are as follows. The signal enhancement structure 100c of the present embodiment further includes a plurality of nano-dendrimers 120c, and the nanowires 110 are stacked on the nano-dendrimers 120c. The material of the nano-dendrimers 120c is, for example, gold, silver, platinum, other precious metals, or a combination thereof.

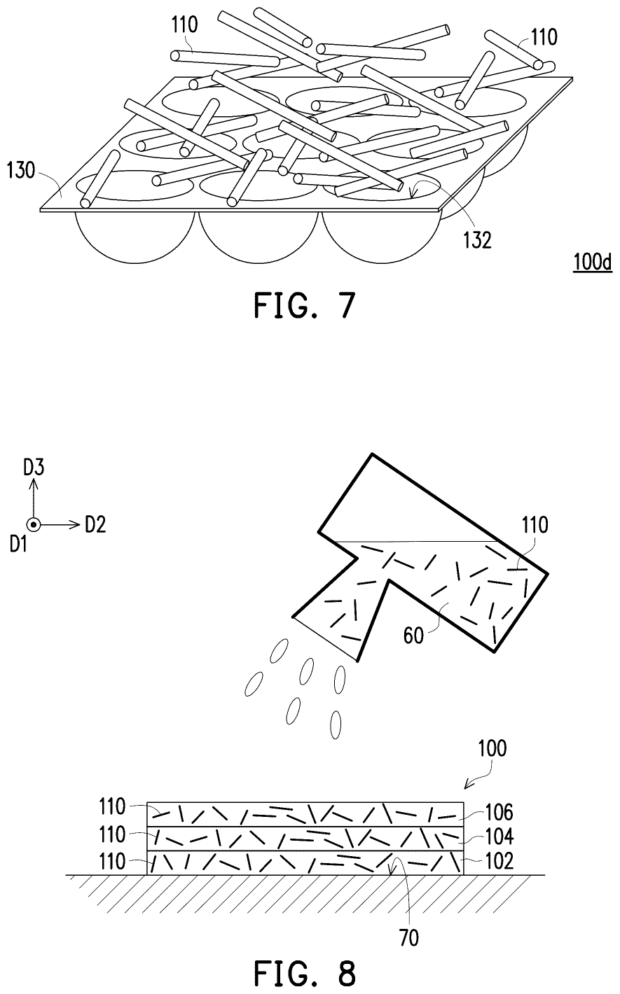

FIG. 7 is a three-dimensional diagram of the signal enhancement structure of another embodiment of the invention. Referring to FIG. 7, a signal enhancement structure 100d of the present embodiment is similar to the signal enhancement structure 100 of FIG. 2A, and the differences between the two are as follows. The signal enhancement structure 100d of the present embodiment further includes a nanostructure chip 130, and the nanowires 110 are disposed on the nanostructure chip 130. The surface of the nanostructure chip 130 may have nanostructures 132. In the present embodiment, the nanostructures 132 facing the nanowires 110 are, for example, nano recesses. However, in other embodiments, the nanostructures 132 facing the nanowires 110 may also be nano bumps, or a combination of nano recesses and nano bumps. The nanostructure chip 130 is, for example, a titanium dioxide chip, a titanium dioxide-platinum chip, or a gold nanochip.

FIG. 8 is a cross-sectional view for explaining the manufacturing method of a signal enhancement structure of an embodiment of the invention. Referring to FIG. 2A and FIG. 8, the manufacturing method of the signal enhancement structure of the present embodiment may be used to manufacture a signal enhancement structure of the above embodiment (such as the signal enhancement structure 100). The manufacturing method of the signal enhancement structure of the present embodiment includes the following steps. First, as shown in FIG. 8, the plurality of nanowires 110 dispersed in a solvent 60 are sprayed on a surface 70 to form a first nanowire layer 102. Then, after the solvent 60 in the first nanowire layer 102 is volatilized, the plurality of nanowires 110 dispersed in the solvent 60 are sprayed on the first nanowire layer 102 again to form a second nanowire layer 104. After the solvent of the second nanowire layer 104 is volatilized, the signal enhancement structure 100 as shown in FIG. 2A may be formed. In a general embodiment, after the solvent in the second nanowire layer 104 is volatilized, the plurality of nanowires 110 dispersed in the solvent 60 may be sprayed on the second nanowire layer 104 again to form a third nanowire layer 106. In this way, after the solvent 60 of the third nanowire layer 106 is volatilized, a thicker signal enhancement structure 100 may be formed. The number of the nanowire layer is not limited to two or three layers as above. In other embodiments, only one layer or N layers may be sprayed, wherein N is a positive integer greater than or equal to 2. In another embodiment of the invention, N preferably ranges from 2 to 5.

The surface 70 may be the surface of any object, or the surface of a specimen. When the surface 70 is the surface of the specimen, the above nanowire layer is sprayed on the surface 70, wherein the nanowire layer spray is a single layer. In another embodiment of the invention, the nanowire layer spray may be a plurality of layers, and the preferred number is two layers. After the solvent 60 is volatilized, a laser beam may be irradiated on the surface 70, and then the particles 50 of the surface 70 convert the laser beam into converted light beam, which is detected to obtain the Raman signal or photoluminescence signal of the specimen. Therefore, the signal enhancement structure 100 enhances the Raman signal or the photoluminescence signal of the particles 50. When the surface 70 is the surface of a carrier board or the surface of any carrier (for example, it may also be the surface of the nanostructure chip 130 shown in FIG. 7), after the above nanowire layers are sprayed on the surface 70 and the solvent 60 is volatilized, the specimen may be placed on the surface 70, added dropwise on the surface 70, coated on the surface 70, or disposed on the surface 70 in any suitable form. In another embodiment of the invention, the number of the nanowire layer sprayed is preferably 2 to 5 layers. After that, the surface 70 is irradiated with a laser beam as described above to obtain a Raman signal or a photoluminescence signal.

In addition, in the manufacture of the signal enhancement structure 100b of FIG. 5, the plurality of nanoparticles 120 may be mixed into the solvent 60, and then the plurality of nanoparticles 120 are sprayed on the surface 70. Similarly, in the manufacture of the signal enhancement structure 100c of FIG. 6, a plurality of nano-dendrimers 120c may be mixed into the solvent 60, and then the plurality of nano-dendrimers 120c are sprayed on the surface 70.

Since the signal enhancement structure of each of the above embodiments may simultaneously enhance the Raman spectrum and the photoluminescence spectrum (such as fluorescence spectrum), if the specimen has a photoluminescence spectrum in addition to the Raman spectrum, then the signal enhancement structure of each of the embodiments above may be used to simultaneously measure the Raman spectrum and the photoluminescence spectrum of the specimen. A spectrum measurement system simultaneously measuring the two spectra is described as follows.

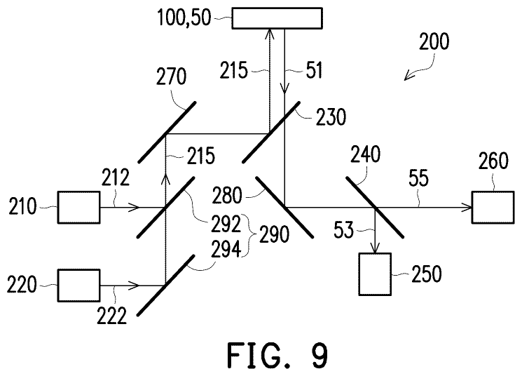

FIG. 9 is a diagram of the optical path architecture of a spectrum measurement system of an embodiment of the invention. Referring to FIG. 9, a spectrum measurement system 200 of the present embodiment includes a first laser light source 210, a second laser light source 220, a light combining unit 290, a beam splitter 230, a dichroic mirror 240, a first light detection module 250, and a second light detection module 260. The first laser light source 210 is configured to emit a first peak wavelength laser beam 212, and the second laser light source 220 is configured to emit a second peak wavelength laser beam 222, wherein the first peak wavelength of the first peak wavelength laser beam 212 is greater than the second peak wavelength of the second peak wavelength laser beam 222. The first peak wavelength laser beam 212 is configured to measure the Raman spectrum of the particles 50 of the specimen, and the second peak wavelength laser beam 222 is configured to measure the photoluminescence spectrum of the particles 50 of the specimen. The light combining unit 290 combines the first peak wavelength laser beam 212 and the second peak wavelength laser beam 222 into a laser output beam 215. In the present embodiment, the light combining unit 290 may include a dichroic mirror 292 and a dichroic minor or reflector 294. The dichroic mirror or reflector 294 reflects the second peak wavelength laser beam 222 to the dichroic mirror 292. The dichroic mirror 292 is suitable for reflecting the first peak wavelength laser beam 212 and is suitable for allowing the second peak wavelength laser beam 222 to penetrate, thus combining the first peak wavelength laser beam 212 and the second peak wavelength laser beam 222.

The beam splitter 230 reflects the laser output beam 215 to the particles 50 of the specimen and the signal enhancement structure 100. In the present embodiment, the spectrum measurement system 200 may further include a reflector 270 to reflect the laser output beam 215 to the beam splitter 230. The particles 50 of the specimen converts the laser output beam 215 into a converted beam 51, wherein the converted beam 51 contains a Raman signal beam and a photoluminescence signal beam. A portion of the converted light beam 51 penetrates the beam splitter 230 and is transmitted to the dichroic mirror 240. In the present embodiment, the spectrum measurement system 200 may further include a reflecting mirror 280 to reflect the converted beam 51 from the beam splitter 230 to the dichroic minor 240. The dichroic mirror 240 reflects a portion of the converted beam 53 in the converted beam 51 corresponding to the Raman signal to the first light detection module 250, and allows a portion of the converted beam 55 in the converted beam 51 corresponding to the photoluminescence signal to penetrate to be transmitted to the second light detection module 260. In this way, the first light detection module 250 may detect the Raman spectrum, and the second light detection module 260 may detect the photoluminescence spectrum, so that the spectrum measurement system 200 may achieve the simultaneous detection of Raman spectrum and photoluminescence spectrum. Each of the first light detection module 250 and the second light detection module 260 may sequentially include a light filter and a light detector along the path of the light transmission direction. In addition, in other embodiments, the first laser light source 210 may also emit the first peak wavelength laser beam 212 so that the second laser light source 220 does not emit the second peak wavelength laser beam 222, so that the spectrum measurement system 200 achieves the effect of measuring Raman spectrum without also measuring photoluminescence spectrum. Alternatively, the first laser light source 210 may not emit the first peak wavelength laser beam 212, and the second laser light source 220 may emit the second peak wavelength laser beam 222, so that the spectrum measurement system 200 achieves the effect of measuring photoluminescence spectrum without also measuring Raman spectrum.

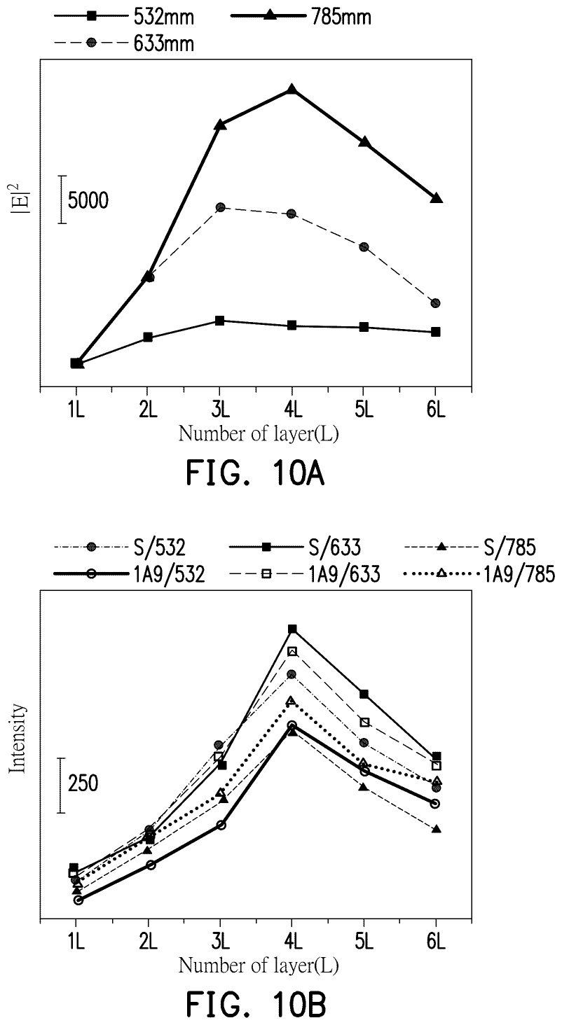

FIG. 10A and FIG. 10B respectively show the theoretical electric field values and experimental SERS results of multilayer nanostructured silver nanowires. FIG. 10A is made by light sources with wavelengths of 532 nm, 633 nm, and 785 nm. FIG. 10B is made by light sources with wavelengths of 532 nm, 633 nm, and 785 nm using two specimen denoted by “S” and “1A9”. In FIG. 10A, finite integration technique (FIT) simulated electric field values show the existence of an optimum irrespective of incident irradiation wavelength sources, which is which is also well supported by the experimental SERS measurements of 2-dimensional silver nanowire layers with different biomolecules under the same conditions as shown in FIG. 10B. In FIG. 10A, |E|² at the vertical axis is electromagnetic field, which is a theoretical computational value. In FIG. 10B, the unit of the vertical axis is photon counting or SERS intensities, the intensity at the vertical axis means the photon counts detected by the sensor such as a charge coupled device (CCD).

It can be learned from FIG. 10A and FIG. 10B that the number of nanowire layers which most enhances the signal is about 4. If the diameter of a nanowire is about 100 nm, the thickness of the film layer formed by nanowires which most enhances the signal ranges from 350 nm to 550 nm.

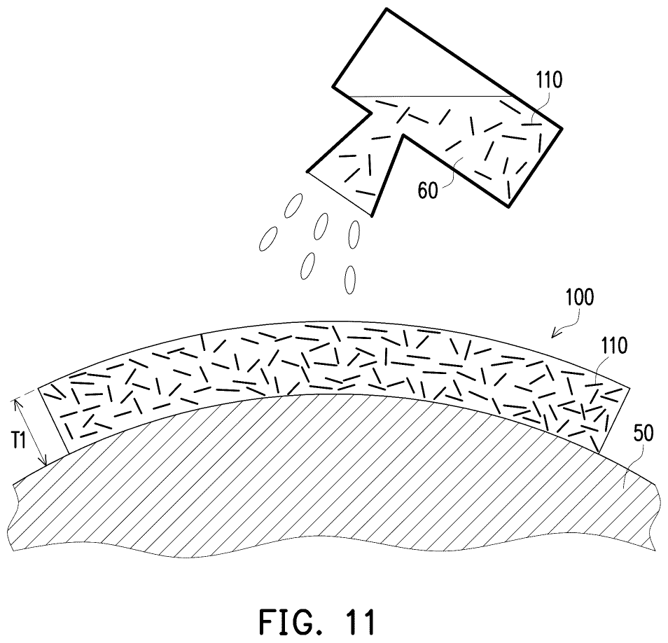

FIG. 11 is a schematic structural view showing a step of a method with signal enhancement according to an embodiment of the invention. Referring to FIG. 9 and FIG. 11, the measuring method with signal enhancement in this embodiment includes the following step. First, referring to FIG. 11, a plurality of nanowires 110 dispersed in a solvent 60 are dropped or sprayed on a surface of a specimen 50 to form a film layer on the specimen 50, wherein the film layer has a thickness T1 ranging from 350 nanometers to 550 nanometers. Next, referring to FIG. 9, at least one of a Raman spectrum and a photoluminescence spectrum of the specimen 50 is measured, a signal of which is enhanced by the nanowires 110 as described on the above. The specimen 50 may be any kind of specimen such as a fruit.

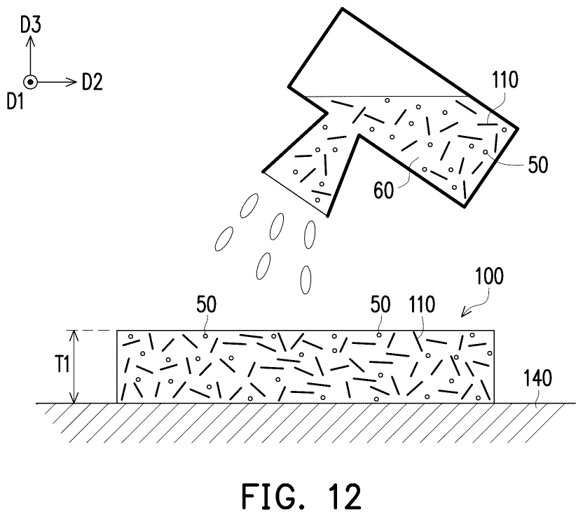

FIG. 12 is a schematic structural view showing a step of a method with signal enhancement according to another embodiment of the invention. Referring to FIG. 9 and FIG. 12, the measuring method with signal enhancement in this embodiment includes the following steps. First, referring to FIG. 12, a specimen 50 is mixed with a plurality of nanowires 110 dispersed in a solvent 60. Next, the specimen 50 with the nanowires 110 dispersed in the solvent 60 is dropped or sprayed onto a substrate 140 to form a film layer on the substrate 140, wherein the film layer has a thickness T1 ranging from 350 nanometers to 550 nanometers. Then, referring to FIG. 9 at least one of a Raman spectrum and a photoluminescence spectrum of the specimen 50 is measured, a signal of which is enhanced by the nanowires 110.

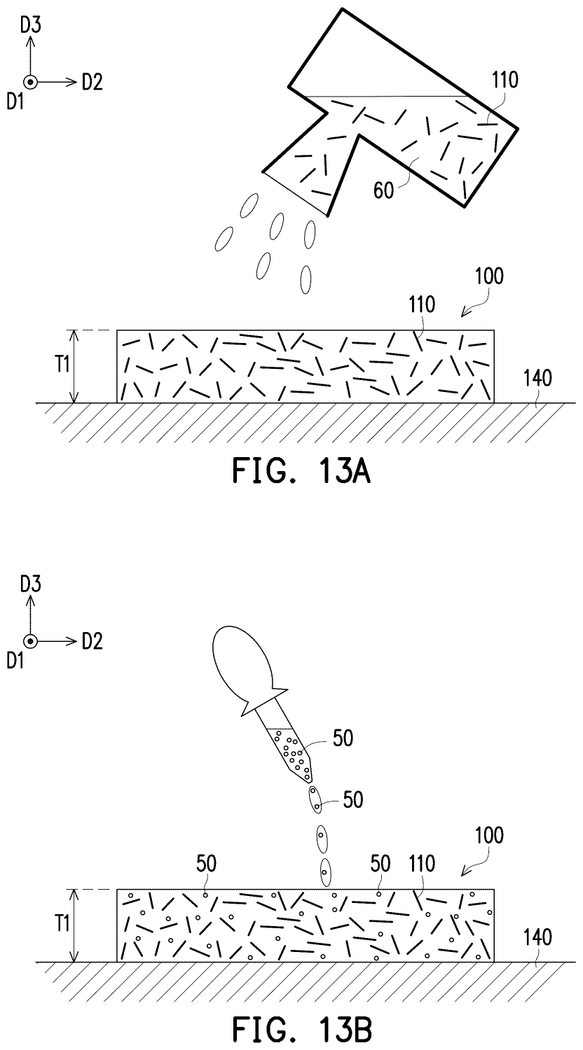

FIG. 13A and FIG. 13B are schematic structural views showing two steps of a method with signal enhancement according to another embodiment of the invention. Referring to FIG. 9, FIG. 13A, and FIG. 13B, the measuring method with signal enhancement in this embodiment includes the following steps. First, referring to FIG. 13A, a plurality of nanowires 110 dispersed in a solvent 60 are dropped or sprayed on a substrate 140 to form a film layer on the substrate 140, wherein the film layer has a thickness T1 ranging from 350 nanometers to 550 nanometers. Next, referring to FIG. 13B, a specimen 50 is dropped or sprayed onto the film layer. Then, at least one of a Raman spectrum and a photoluminescence spectrum of the specimen 50 is measured, a signal of which is enhanced by the nanowires 110. In other embodiments, the nanowires 110 may be dropped or sprayed on nanoparticles 120 as shown in FIG. 5.

In FIG. 11 to FIG. 13B, when measuring the at least one of a Raman spectrum and a photoluminescence spectrum of the specimen 50, the film layer may be solidified or not solidified; that is, the film layer may have solvent 60 or may not have solvent 60. In an embodiment, when forming the film layer, the nanowires 110 with the solvent 60, such as water, may be sprayed or dropped 4 times, and 20 micro-liter thereof may be sprayed or dropped per time. In another embodiment, 60 micro-liter of the nanowires 110 with the solvent 60, such as water, may be sprayed or dropped with the nanowires 110 having 0.6 optical density (OD).

Based on the above, in the signal enhancement structure of an embodiment of the invention, since the nanowires are stacked in the first direction, the second direction, and the third direction, the particles of the specimen may have different distances from different nanowires, such that the signal of the particles of the specimen may be enhanced. Therefore, the signal enhancement structure of an embodiment of the invention is suitable for particles of a specimen having different sizes. In the manufacturing method of a signal enhancement structure of an embodiment of the invention, since the nanowires are sprayed on the surface a plurality of times along with the solvent, the particles of the specimen may have different distances from different nanowires, such that the signal of the particles of the specimen may be enhanced. Therefore, the manufacturing method of the signal enhancement structure of an embodiment of the invention is suitable for particles of a specimen having different sizes. In the signal enhancement structure and the measuring method with signal enhancement according to the embodiment of the invention, since the thickness of the film layer ranges from 350 nanometers to 550 nanometers, the signal enhancement is more significant.

What is claimed is:

1. A signal enhancement structure configured to enhance a signal of a specimen, the signal enhancement structure comprising:
a plurality of nanowires stacked in a first direction, a second direction, and a third direction, wherein the nanowires are extended along at least two directions, an included angle of the nanowires is varied in planes perpendicular to the first direction, the second direction, and the third direction, and a particle of the specimen is on the nanowires or in a gap among the nanowires or the nanowires are on the specimen, and
wherein the nanowires are stacked in the third direction to form a film layer, the third direction is a thickness direction of the film layer, the first direction and the second direction are both perpendicular to the third direction, and a thickness of the film layer in the third direction ranges from 350 nanometers to 550 nanometers.

2. The signal enhancement structure of claim 1, wherein the particle of the specimen has different distances from different nanowires in the third direction.

3. The signal enhancement structure of claim 1, wherein a ratio of a width of a largest gap to a smallest gap among the nanowires is within a range of 50 to 2000.

4. The signal enhancement structure of claim 1, further comprising a plurality of nanoparticles, wherein the nanowires are stacked on the nanoparticles.

5. The signal enhancement structure of claim 1, further comprising a plurality of nano-dendrimers, wherein the nanowires are stacked on the nano-dendrimers.

6. The signal enhancement structure of claim 1, wherein the nanowires are irregularly distributed.

7. The signal enhancement structure of claim 1, wherein the nanowires are regularly distributed.

8. The signal enhancement structure of claim 1, wherein the nanowires have a curved shape, a straight shape, or a combination thereof.

9. The signal enhancement structure of claim 1, further comprising a nanostructure chip, wherein the nanowires are disposed on the nanostructure chip.

10. The signal enhancement structure of claim 1, wherein a material of the nanowires comprises gold, silver, platinum, or a combination thereof.

11. A measuring method with signal enhancement comprising:
dropping or spraying a plurality of nanowires dispersed in a solvent on a surface of a specimen to form a film layer on the specimen, wherein the film layer has a thickness ranging from 350 nanometers to 550 nanometers; and
measuring at least one of a Raman spectrum and a photoluminescence spectrum of the specimen, a signal of which is enhanced by the nanowires.

12. The measuring method with signal enhancement of claim 11, wherein the nanowires in the film layer are stacked in a first direction, a second direction, and a third direction, wherein the nanowires are extended along at least two directions, an included angle of the nanowires is varied in planes perpendicular to the first direction, the second direction, and the third direction.

13. The measuring method with signal enhancement of claim 12, wherein in the film layer, an included angle of the nanowires is varied in planes perpendicular to the first direction, the second direction, and the third direction.

14. The measuring method with signal enhancement of claim 11, wherein in the film layer, a ratio of a width of a largest gap to a smallest gap among the nanowires is within a range of 50 to 2000.

15. The measuring method with signal enhancement of claim 11, further comprising spraying a plurality of nanoparticles on the specimen, wherein in the film layer, the nanowires are stacked on the nanoparticles.

16. The measuring method with signal enhancement of claim 11, wherein in the film layer, the nanowires are irregularly distributed.

17. The measuring method with signal enhancement of claim 11, wherein in the film layer, the nanowires have a curved shape, a straight shape, or a combination thereof.

18. The measuring method with signal enhancement of claim 11, wherein a material of the nanowires comprises gold, silver, platinum, or a combination thereof.

19. A measuring method with signal enhancement comprising:
mixing a specimen with a plurality of nanowires dispersed in a solvent;
dropping or spraying the specimen with the nanowires dispersed in the solvent onto a substrate to form a film layer on the substrate, wherein the film layer has a thickness ranging from 350 nanometers to 550 nanometers; and
measuring at least one of a Raman spectrum and a photoluminescence spectrum of the specimen, a signal of which is enhanced by the nanowires.

20. The measuring method with signal enhancement of claim 19, wherein in the film layer, a ratio of a width of a largest gap to a smallest gap among the nanowires is within a range of 50 to 2000.

21. A measuring method with signal enhancement comprising:
dropping or spraying a plurality of nanowires dispersed in a solvent on a substrate to form a film layer on the substrate, wherein the film layer has a thickness ranging from 350 nanometers to 550 nanometers;
dropping or spraying a specimen onto the film layer; and
measuring at least one of a Raman spectrum and a photoluminescence spectrum of the specimen, a signal of which is enhanced by the nanowires.

22. The measuring method with signal enhancement of claim 21 further comprising spraying a plurality of nanoparticles on the substrate, wherein in film layer, the nanowires are stacked on the nanoparticles.
